(19) United States
(12) Patent Application Publication
(10) Pub. No.: US 2026/0198878 A1
(43) Pub. Date: Jul. 16, 2026

(54) Combined Imaging Apparatus with a Position-Determining Unit

(71) Applicant: Siemens Healthineers AG, Forchheim (DE)

(72) Inventors: Johann Sukkau, Herzogenaurach (DE); Steffen Schröter, Fürth (DE)

(73) Assignee: Siemens Healthineers AG, Forchheim (DE)

(21) Appl. No.: 19/448,160

(22) Filed: Jan. 14, 2026

(30) Foreign Application Priority Data
Jan. 15, 2025 (DE) 10 2025 101 247.4

(51) Int. Cl. A61B 6/00; A61B 5/00; A61B 5/055; A61B 6/03; A61B 6/04; G01R 33/30; G01R 33/421; G01R 33/48; G06T 7/70; G06T 12/10

(52) U.S. Cl. CPC A61B 6/4423; A61B 5/0033; A61B 5/0035; A61B 5/055; A61B 5/704; A61B 6/037; A61B 6/0407; A61B 6/5247; G01R 33/307; G01R 33/421; G01R 33/481; G06T 7/70; G06T 12/10; A61B 2562/18; A61B 2562/245; G06T 2200/24; G06T 2207/10088; G06T 2207/10104; G06T 2210/41

(57) ABSTRACT

A combined imaging apparatus is described that includes a first medical imaging apparatus, which is embodied as a magnetic resonance apparatus, and a further medical imaging apparatus, with a patient-receiving region, a patient-supporting apparatus, which has a patient table which can be moved in at least one direction, and a position-determining system, which is embodied to determine a position of the patient table in at least one direction and which has a capturing unit, wherein the position-determining system has a shielding system for an arrangement of the capturing unit, wherein the shielding system has a shielding housing with an optically transparent viewing window and a protective cover for the optically transparent viewing window.

### CROSS-REFERENCE TO RELATED APPLICATIONS

The present application claims priority to and the benefit of Germany patent application no. DE 10 2025 101 247.4, filed on Jan. 15, 2025, the contents of which are incorporated herein by reference in their entirety.

### TECHNICAL FIELD

The present disclosure relates to a combined imaging apparatus, comprising a first medical imaging apparatus, which is embodied as a magnetic resonance apparatus, and a further medical imaging apparatus, with a patient-receiving region, a patient-supporting apparatus, which has a patient table which can be moved in at least one direction, and a position-determining unit, which is embodied to determine a position of the patient table in at least one direction and which has a capturing unit.

### BACKGROUND

With combined imaging apparatuses, for example an imaging apparatus which has a magnetic resonance apparatus and a PET apparatus (Positron Emissions Tomography apparatus) or a magnetic resonance apparatus and an X-ray apparatus, it is important that an exact position of the patient table and therewith of the region of the patient to be examined exists for the different imaging examinations. It is desirable to obtain the position of the patient table and therewith, in particular, of the region of the patient to be examined in more than just one spatial direction. For this, it is customary that a combined imaging apparatus has a position-determining unit.

A position-determining unit of this kind can be arranged at least partially inside the patient-receiving region of the combined imaging apparatus. The position-determining unit is arranged underneath the patient table to prevent an obstruction of the patient by the position-determining unit. In addition, the position-determining unit can also be arranged so as to be particularly protected in this way. For example, the position-determining unit can have a capturing unit for capturing the position of the patient table, wherein the capturing unit is arranged underneath the patient table inside the patient-receiving region, in particular on an enclosure surrounding the patient-receiving region. The capturing unit may be directed at the patient table for capturing the position of the patient table. However, an arrangement of the capturing unit of this kind has the risk that dirt can be deposited on the capturing unit or liquids can also penetrate into the capturing unit.

### SUMMARY

The present disclosure is directed to providing advantageous protection for a position-determining unit arranged on the enclosure of the patient-receiving region. The object is achieved by the features of the embodiments as discussed herein, including the claims.

The disclosure describes a combined imaging apparatus which comprises a first medical imaging apparatus, wherein the first medical imaging apparatus is embodied as a magnetic resonance apparatus, and a further medical imaging apparatus, with a patient-receiving region, a patient-supporting apparatus, which has a patient table which can move in at least one direction, and a position-determining unit which is embodied to determine a position of the patient table in at least one direction and which has a capturing unit. Inventively, the position-determining unit has a shielding unit for an arrangement of the capturing unit, wherein the shielding unit has a shielding housing with an optically transparent viewing window and a protective cover for the optically transparent viewing window.

The combined imaging apparatus may e.g. comprise two different imaging medical modalities and/or two different medical imaging apparatuses, which are embodied for different examinations of a region of a patient to be examined. The first medical modality and/or the first medical imaging apparatus comprises a magnetic resonance apparatus. The second medical modality and/or the second medical imaging apparatus comprises a medical modality different from a magnetic resonance apparatus and/or different medical imaging apparatus. The second medical modality and/or the second medical imaging apparatus can comprise a PET apparatus or an X-ray apparatus and/or further medical imaging apparatuses which appear expedient to a person skilled in the art.

The magnetic resonance apparatus may e.g. comprise a medical and/or diagnostic magnetic resonance apparatus, which is designed and/or embodied to capture medical and/or diagnostic image data, e.g. medical and/or diagnostic magnetic resonance image data, of a patient. The magnetic resonance apparatus comprises for this a scanner unit embodied as a magnetic unit for capturing the medical and/or diagnostic image data. In an embodiment, the magnetic unit comprises here a main magnet, a gradient unit, and a radio-frequency antenna unit. The radio-frequency antenna unit may e.g. be permanently arranged inside the magnetic unit and designed and/or embodied to emit excitation pulses, e.g. radio-frequency pulses.

The main magnet is embodied to generate a homogeneous main magnetic field with a defined magnetic field strength, such as with a magnetic field strength of 3 T or 1.5 T, etc. For example, the main magnet may be embodied to generate a strong and constant main magnetic field. The homogeneous main magnetic field may e.g. be arranged and/or be found inside the patient-receiving region of the magnetic resonance apparatus. The gradient unit may be configured to generate magnetic field gradients, which are used for spatial encoding during imaging.

The PET apparatus comprises a plurality of Positron Emission Tomography detector modules (PET detector modules) which may e.g. be arranged in an annular shape and surround the patient-receiving region in a circumferential direction. The PET detector modules can each have a plurality of Positron Emission Tomography detector elements (PET detector elements), which are arranged to form a PET detector array that comprises a scintillation detector array with scintillation crystals, for example LSO crystals. Furthermore, the PET detector modules may e.g. be each comprise a photodiode array, for example Avalanche photodiode array or APD photodiode array, which are arranged inside the PET detector modules connected downstream of the scintillation detector array.

Photon pairs, which result from the annihilation of a positron with an electron, are captured by means of the PET detector modules. The positron is emitted here by a radiopharmaceutical, with the radiopharmaceutical being administered to the patient via an injection. Trajectories of the two photons enclose an angle of 180°. In addition, the two photons each have an energy of 511 keV. When passing through material in the beam path, the PET photons that developed during the annihilation can be attenuated, with the attenuation depending on the path length through material and the corresponding attenuation coefficient of the respective material.

The X-ray apparatus may e.g. be an X-ray source and an X-ray detector and be embodied to record X-ray image data of an organ or body part of the patient.

The two medical imaging apparatuses may e.g. be embodied in such a way that a scanner unit of the second medical imaging apparatus is integrated in a scanner unit of the magnetic resonance apparatus and only one single device with a single patient-receiving region for an examination of the patient, may of the region of the patient to be examined, is available. In addition, the two medical imaging apparatuses may e.g. comprise two separate devices, with the patient having to be re-positioned between the two medical devices for an examination.

The patient-receiving region may e.g. be designed and/or embodied for a recording of the patient, e.g. of the region of the patient to be examined, for a medical magnetic resonance examination and a further medical imaging examination. The patient-receiving region may also comprise the region which is available to the patient during the medical imaging examinations. For example, the patient-receiving region may be cylindrical for this purpose and/or be cylindrically surrounded by the magnetic unit of the magnetic resonance apparatus and/or cylindrically surrounded by the scanner unit of the further imaging apparatus. In an embodiment, the magnetic unit and/or the scanner unit of the further imaging apparatus comprises an enclosure which at least partially surrounds the patient-receiving region. The enclosure may surround the patient-receiving region cylindrically.

A Field of View (FOV) and/or an isocenter of the combined imaging apparatus may e.g. be arranged inside the patient-receiving region. The FOV may e.g. comprise a capture range of the magnetic resonance apparatus and/or the further imaging apparatus, within which the conditions for capturing medical image data, for example magnetic resonance image data and/or PET image data and/or X-ray image data, are present inside the patient-receiving region. For example, the FOV may comprise a homogeneous main magnetic field of a magnetic resonance apparatus and/or a field of vision of the PET detector modules and/or a field of vision of an X-ray detector. The isocenter of the combined imaging apparatus may e.g. comprise the region and/or point inside the combined imaging apparatus, which has the optimum and/or ideal conditions for capturing medical image data. For example, the isocenter may comprise the most homogeneous magnetic field region inside the magnetic resonance apparatus. In addition, the isocenter may also comprise a center and/or a center point of the field of vision of the PET apparatus and/or of the field of vision of the X-ray apparatus.

For a positioning of the patient, e.g. of the region of the patient to be examined inside the patient-receiving region, the combined imaging apparatus may have the patient-supporting apparatus. The patient-supporting apparatus may e.g. be embodied to support the patient during the combined medical imaging examination. The patient-supporting apparatus may e.g. have a movable patient table which is embodied so it can move, for instance, inside the patient-receiving region of the combined imaging apparatus. In an embodiment, the patient table is embodied so it can move inside the patient-receiving region in the longitudinal direction of the patient-receiving region. The longitudinal direction of the patient-receiving region may e.g. be oriented parallel to a z-direction of the combined imaging apparatus. When the patient table is retracted into the patient-receiving region, the patient table is guided on a guide rail in the z-direction. The movement may have any suitable tolerance in the x-direction of the combined imaging apparatus, such as for example approximately 1 mm. The x-direction of the combined imaging apparatus may be oriented perpendicular to the z-direction and perpendicular to a weight force acting on the patient table. Furthermore, with a positioning of a heavy patient on the patient table, the patient table may also sag more than with a positioning of a light patient on the patient table or an empty patient table. Thus, a position of the patient table may also vary in the y-direction of the combined imaging apparatus. The y-direction of the combined imaging apparatus is oriented perpendicular to the z-direction and perpendicular to the x-direction of the combined imaging apparatus. In an embodiment, the y-direction of the combined imaging apparatus may be oriented parallel to the weight force acting on the patient table. The x-direction, the y-direction, and the z-direction may be oriented orthogonal to one another.

For determining a position of the patient table in at least one direction, the combined imaging apparatus includes the position-determining unit. The position of the patient table may e.g. be determined with respect to a reference point that is encompassed by the patient-receiving region and/or at least one of the scanner units. For example, the reference point may comprise an opening of the patient-receiving region. Alternatively or additionally, the reference point may also comprise the isocenter of the magnetic resonance apparatus and/or the isocenter of the further imaging apparatus.

The position-determining unit may include the capturing unit. The capturing unit may e.g. be embodied to capture position information and/or position data of the patient table. In an embodiment, the capturing unit has for this purpose a sensor unit, for example a camera and/or an optical sensor unit. The capturing unit may e.g. be arranged on the enclosure surrounding the patient-receiving region so when the patient table moves, there is a relative movement between the position-encoding unit and the capturing unit. Advantageously, the capturing unit may be arranged on the enclosure surrounding the patient-receiving region, in a floor region of the patient-receiving region. In an embodiment, a capture range of the capturing unit is directed at the patient table. The position-determining unit may also comprise a position information element that is arranged on the patient table. The position information element may e.g. be arranged on the patient table in such a way that the position information element is arranged in a field of vision and/or a capture range of the capturing unit. The position information element can comprise e.g. a measuring tape and/or any suitable position encoding, for example a barcode, etc. In addition, further embodiments of the position information element are conceivable.

For an arrangement of the capturing unit inside the patient-receiving region, the position-determining unit includes the shielding unit with a shielding housing. The shielding housing shields the capturing unit with respect to radio-frequency radiation. For capturing items of position information of the patient table, the shielding housing has the optically transparent viewing window. In an embodiment, the optically transparent viewing window, e.g. the transparent cover, also has shielding properties with respect to radio-frequency radiation. In addition, the shielding housing has the protective cover to protect the optically transparent viewing window. In an embodiment, the protective cover is embodied so as to be compatible with magnetic resonance.

This can provide advantageous protection of the position-determining unit. In an embodiment, the protective cover can reduce and/or impede undesirable deposits of dirt on the optically transparent viewing window.

In an advantageous development of the combined imaging apparatus, it can be provided that the protective cover is arranged so it can move on the shielding housing to cover the optically transparent viewing window. Due to the movable arrangement of the protective cover, it is possible for it to be removed from the optically transparent viewing window of the shielding housing during capture of position data and/or items of position information of the patient table. When the position-determining unit is not being used, e.g. when the capturing unit of the position-determining unit arranged inside the shielding housing is not being used, the protective cover can cover the optically transparent viewing window.

The protective cover can advantageously protect the position-determining unit, e.g. the capturing unit, when it is not being used, from dirtying and/or damage. For example, the position-determining unit, e.g. the capturing unit arranged inside the shielding housing, can be protected against dirtying when the patient table is arranged completely outside of the patient-receiving region, and thus the shielding housing with the capturing unit is arranged in a floor region of the patient-receiving region so as to be easily accessible.

In an advantageous development of the combined imaging apparatus, it can be provided that the protective cover is embodied so it can move between two end positions, In the first end position, the protective cover covers the optically transparent viewing window, and in the second end position the optically transparent viewing window is not covered by the protective cover. In this way, a covering position of the protective cover and an open position of the protective cover in respect of the optically transparent viewing window can be set particularly easily and safely. In an embodiment, in the first end position, e.g. in the covering position, the protective cover completely covers the optically transparent viewing window. Similarly, in the second end position, e.g. in the open and/or uncovered position, the optically transparent viewing window of the protective cover is embodied to be completely open. In particular, it is possible in this way to ensure that when an end position is reached, the protective cover completely covers the optically transparent viewing window or completely exposes it for capturing position data and/or items of position information of the patient table.

In an advantageous development of the combined imaging apparatus, it can be provided that the protective cover comprises a cleaning lip, and the cleaning lip is arranged on an edge of the protective cover that faces the optically transparent viewing window. This embodiment enables simple and effective cleaning of the optically transparent viewing window when the protective cover moves from one end position into the second end position. In an embodiment, the cleaning lip is arranged on an edge of the protective cover here, and with a movement of the protective cover from one end position into the second end position, moves on the optically transparent viewing window. The edge of protective cover may for instance extend orthogonally to the direction of movement of the protective cover. The cleaning lip may for instance comprise a rubber material.

In an advantageous development of the combined imaging apparatus, it can be provided that the shielding unit comprises a magnetic resonance-compatible actuator unit that is embodied to generate a drive moment for a movement of the protective cover on the shielding housing. By means of the actuator unit, it is advantageously possible for an automated movement of the protective cover to be provided for a measuring operation of the combined imaging apparatus. In an embodiment, the magnetic resonance-compatible embodiment of the actuator unit can enable an arrangement of the actuator unit inside the patient-receiving region and therewith provide a particularly compact shielding unit. For instance, with an embodiment of this kind, a force transfer path between the magnetic resonance-compatible actuator unit and the protective cover can be kept particularly compact and short.

In one advantageous development of the combined imaging apparatus, it can be provided that the magnetic resonance-compatible actuator unit has a magnetic resonance-compatible stepper motor with an actuator coil, and the actuator coil executes a movement within a magnetic field of the magnetic resonance apparatus as soon as an electric current flows through actuator coil. The magnetic resonance-compatible stepper motor can be embodied here as an electric motor in which the magnetic field of the magnetic resonance apparatus functions as the stator of the electric motor. The actuator coil may be positioned inside the magnetic field in such a way that as soon as an electric current flows through the actuator coil, a Lorentz force acting on the actuator coil causes a movement of the actuator coil. A direction of the movement executed by the actuator coil is thus dependent on a flow direction of the electric current flowing through the actuator coil. By changing the direction of current in the actuator coil, a movement of the actuator coil in the opposite direction is also induced. A current direction can be set and/or the current direction of a current flowing through the actuator coil can be changed, for instance via an H-bridge circuit of the actuator unit. The actuator coil can comprise a plurality of windings made of copper wire, which are arranged so as to be embedded in a housing made of plastics material.

In an embodiment, the actuator unit has at least one force transfer element, and the at least one force transfer element is embodied to transfer a drive force from the actuator coil to the protective cover. The at least one force transfer element can comprise for instance a joint rod and/or further force transfer elements which appear expedient to a person skilled in the art.

In a manner which is simple in terms of construction, this embodiment of the disclosure can provide a magnetic resonance-compatible actuator unit to generate the drive moment for the movement of the protective cover. In addition, a particularly compact actuator unit can also be provided in this way, which does not require an additional stator.

In an advantageous development of the combined imaging apparatus, it can be provided that the actuator coil has the form of a circle segment, and a circle with a radius is assigned to the circle segment, with the radius of the circle segment being smaller than a radius of a circular cross-sectional area of the patient-receiving region. In an embodiment, the actuator coil is shaped in such a way and/or has a size that with a movement, e.g. a rotation, of the actuator coil about an axis of rotation, the actuator coil does not touch and/or has no contact with the enclosure surrounding the patient-receiving region. In an embodiment, the value of the radius of the circle segment comprises, at most, 50% of the value of the radius of the patient-receiving region. In an embodiment, the value of the radius of the circle segment may be any suitable proportionate value, e.g. between 25% and 50%, of the value of the radius of the patient-receiving region. The circle segment of the actuator coil may for instance comprise a central angle of, at most, 90°. In this way, a particularly compact actuator coil, which is adjusted to the spatial conditions of the patient-receiving region, can be provided to generate a movement of the protective cover.

In an advantageous development of the combined imaging apparatus, it can be provided that the actuator unit has an axis of rotation, about which axis of rotation the actuator coil executes a rotational movement to generate the drive moment, with the axis of rotation being oriented parallel to an x-axis of the magnetic resonance apparatus and/or perpendicular to a direction of movement of the protective cover. An arrangement of the axis of rotation parallel to the x-axis can achieve a particularly compact arrangement of the actuator coil, and therewith of the actuator unit, inside the patient-receiving region, e.g. in a region between a lower side of the patient table and the enclosure surrounding the patient-receiving region. The arrangement of the axis of rotation perpendicular to the direction of movement of the protective cover enables a simple and direct transfer of the drive moment and/or a movement moment from the actuator coil to the protective cover for a movement of the protective cover on the shielding housing.

In an advantageous development of the combined imaging apparatus, it can be provided that the axis of rotation is arranged at an end region of the shielding housing, wherein the axis of rotation is arranged on the shielding housing at an edge region of the shielding housing which faces the enclosure of the patient-receiving region. A particularly compact arrangement of the actuator unit can thus be achieved. A further advantage is that an arrangement of this kind of the axis of rotation provides sufficient space for a movement of the actuator coil to generate the drive moment. In addition, an arrangement of this kind of the axis of rotation of the actuator unit can advantageously prevent hindering of the movement of the patient table when generating the drive moment.

In an advantageous development of the combined imaging apparatus, it can be provided that the position-determining unit has a control unit embodied to control a movement of the protective cover. The control unit can for example advantageously provide an automatic movement of the protective cover. In an embodiment, the control unit can advantageously match a movement of the protective cover in one of the two end positions to a capture mode and/or an operating mode of the capturing unit of the position-determining unit. In this way, manual errors, for example a position-determining measurement in the case of an optically transparent viewing window covered by the protective cover, can also advantageously be prevented.

The control unit may e.g. comprise at least one computing module and/or a processor. Thus, the control unit may be embodied, for instance, to execute computer-readable instructions to execute control of the movement of the protective cover. In an embodiment, the control unit comprises a memory unit, the computer-readable items of information may be stored on the memory unit, and the control unit may be embodied to load the computer-readable items of information from the memory unit and to execute the computer-readable items of information in order to execute control of the movement of the protective cover.

The components of the control unit may be embodied for the most part in the form of executable software components. However, these components may also be partially implemented (for example when particularly fast calculations are involved, in the form of software-assisted hardware components, for example FPGAs or the like.) Similarly, the required interfaces (for example when it is merely a matter of acquiring data from other software components) may be embodied as software interfaces. However, these interfaces may also be embodied as interfaces constructed in terms of hardware, which interfaces are actuated by way of suitable software. Of course, it is also conceivable that a plurality of said components are implemented in a combined manner in the form of an individual software component or software-assisted hardware components.

The control unit may e.g. also be embodied to evaluate the position data and/or items of position information of the patient table captured by the position-determining unit, e.g. the capturing unit of the position-determining unit, and therewith to determine a position of the patient table using the captured position data and/or items of position information of the patient table. For this, the control unit may have an evaluation unit and/or further units that appear expedient to a person skilled in the art. In an embodiment, the control unit also has appropriate evaluation algorithms and/or an appropriate position-determining algorithm for this.

For capturing position data and/or items of position information of the patient table by means of the position-determining unit, e.g. the capturing unit of the position-determining unit, the optically transparent viewing window has to be free of the protective cover. For this purpose, the actuator unit is actuated by the control unit in such a way that the protective cover is moved into the second end position and the viewing protective window is open and/or uncovered. At the same time, the capturing unit is actuated by the control unit in such a way that position data and/or items of position information of the patient table are captured. Once the capture of the position data is complete, the actuator unit is actuated by the control unit in such a way that the protective cover is moved into the first end position and covers the optically transparent viewing window.

The control unit of the position-determining unit may e.g. be arranged outside of the patient-receiving region and, therewith, outside of the shielding housing. Data may be transferred inside the position-determining unit, e.g. between the capturing unit and the control unit, and therewith data be transferred between a region inside the shielding housing, which is arranged inside the patient-receiving region, and a region outside of the patient-receiving region, e.g. via cables. Such cables can comprise, for example, fiber optic cables and/or further cables which appear expedient to a person skilled in the art.

In an advantageous development of the combined imaging apparatus, it can be provided that the control unit is embodied to detect a state (e.g. a viewing state and/or dirtying) of the optically transparent viewing window on the basis of captured position data and/or captured items of position information of the patient table. In an embodiment, the evaluation unit may execute any suitable algorithm to detect and determine a degree of dirtying of the optically transparent viewing window in the captured position data and/or the captured items of position information of the patient table. For example, ideal position data with an ideally clean optically transparent viewing window can be stored in the control unit, e.g. in the evaluation unit, and the evaluation unit may establish and determine a degree of dirtying by way of a comparison of position data currently being captured with the ideal position data. This embodiment of the disclosure has the advantage that dirtying of the optically transparent viewing window can be detected in good time and in the event of dirtying of the optically transparent viewing window, appropriate cleaning measures are initiated and/or are communicated to a user, e.g. a medical operator.

In one advantageous development of the combined imaging apparatus, it can be provided that in the event of dirtying of the optically transparent viewing window detected by the control unit, the control unit is embodied to initiate a cleaning process by way of a movement of the protective cover. Advantageously, automatic cleaning of the optically transparent viewing window can be provided hereby. This can reduce, for instance, the cleaning effort for a medical operator or member of cleaning staff since the optically transparent viewing window is arranged inside the patient-receiving region of the combined imaging apparatus, and is thereby difficult for the medical operator or cleaning staff to access. For example, advantageous cleaning of the optically transparent viewing window can be achieved by attaching a cleaning element, such as, for instance, a cleaning lip, to the protective cover.

In an advantageous development of the combined imaging apparatus, it can be provided that, when dirtying of the optically transparent viewing window is detected, the control unit is embodied to generate user information in respect of the dirtying and provide it for output to a user. In an embodiment, such user information may be provided to an output unit of the combined imaging apparatus, so a user, e.g. a medical operator and/or member of medical cleaning staff, may be informed directly in the event of detected dirtying of the optically transparent viewing window. Apart from information about detected dirtying, the user information can also comprise cleaning information. For example, the user may be asked to start a cleaning process that comprises a cleaning motion of the protective cover. In addition, the user may be asked to manually clean the optically transparent viewing window. The user information may be generated and provided as an alternative (or also in addition) to the cleaning process initiated by the control unit in the event of detected dirtying of the optically transparent viewing window.

In an advantageous development of the combined imaging apparatus, it can be provided that the optically transparent viewing window has a glass cover with an electrically conductive coating. For example, the optically transparent viewing window can be formed from glass, e.g. from a shatter-proof glass. For the shielding properties, the glass may e.g. be transparent and electrically conductive coating. For example, a coating of this kind can be formed from an ITO (indium tin oxide) material. Alternatively or additionally, it is also conceivable that the transparent cover comprises a coating, for example a thin conductive layer made of silver. In an embodiment, by way of the electrically conductive coating being transparent, capturing items of position information and/or position data of the patient table by the capturing unit arranged inside the shielding housing can still be guaranteed.

In an advantageous development of the combined imaging apparatus, it can be provided that the optically transparent viewing window is arranged inclined by at least 10° in respect of a horizontal plane. The horizontal plane describes a plane whose normal vector is oriented parallel to the direction of the weight force (e.g. perpendicular to a vertical plane). The optically transparent viewing window may e.g. be arranged on the shielding housing inclined with respect to the horizontal plane. In an embodiment, the optically transparent viewing window may be arranged inclined by any suitable angle with respect to the horizontal plane, such as for instance at least 15°, at least 18°, at least 20°, at least 22°, etc. For an arrangement of this kind of the optically transparent viewing window, the shielding housing may also be arranged and/or embodied in such a way that a side of the shielding housing comprising the optically transparent viewing window is arranged inclined in respect of the horizontal plane by any suitable angle. In this way, undesirable depositing of dirt on the optically transparent viewing window can advantageously be reduced and/or impeded. In addition, the inclination of the optically transparent viewing window with respect of the horizontal plane can advantageously prevent undesirable penetration of liquids and, therewith, the capturing unit can advantageously be arranged so as to be protected against dirtying and/or damage.

### BRIEF DESCRIPTION OF THE DRAWINGS

Further advantages, features, and details of the disclosure can be found in the exemplary embodiment described below as well as on the basis of the drawings, in which:

FIG. 1 illustrates an example combined imaging apparatus with a patient-supporting apparatus and a position-determining unit, in accordance with one or more embodiments of the present disclosure;

FIG. 2 illustrates a side view of an example shielding housing of the position-determining unit with a protective cover situated in a first end position, in accordance with one or more embodiments of the present disclosure;

FIG. 3 illustrates a plan view of the example shielding housing with the protective cover situated in the first end position, in accordance with one or more embodiments of the present disclosure; and

FIG. 4 illustrates in a perspective view the shielding example housing with the protective cover situated in a second end position, in accordance with one or more embodiments of the present disclosure.

### DETAILED DESCRIPTION OF THE DISCLOSURE

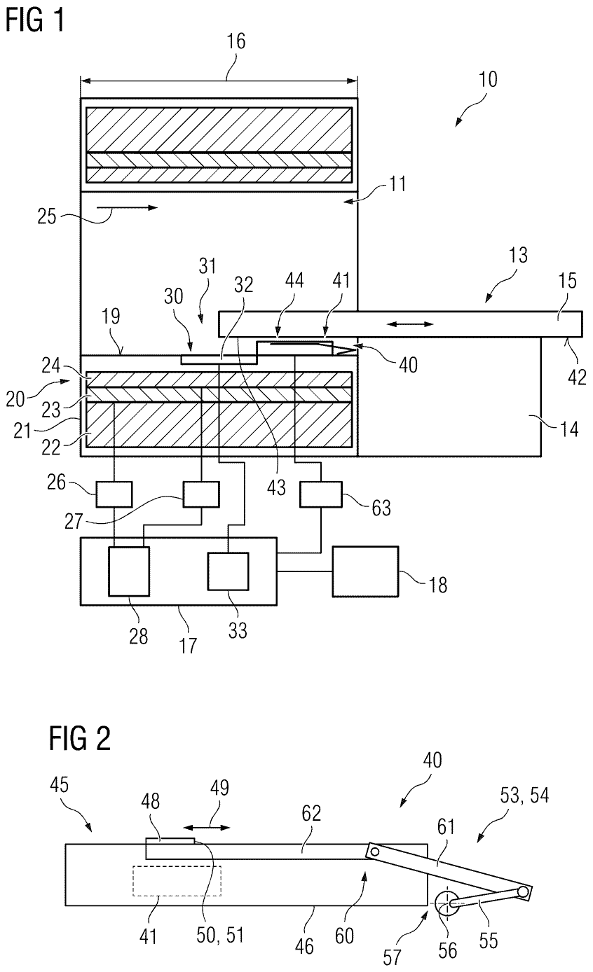

FIG. 1 illustrates an example combined imaging apparatus with a patient-supporting apparatus and a position-determining unit, in accordance with one or more embodiments of the present disclosure. For instance, FIG. 1 schematically represents a combined imaging apparatus 10. The combined imaging apparatus 10 includes a first medical imaging apparatus, which is embodied as a magnetic resonance apparatus 20. The combined imaging apparatus 10 has a further imaging apparatus 30, which in the present exemplary embodiment is embodied as a PET apparatus. However, the present disclosure is not limited to embodiment of the second medical imaging apparatus 30 to a PET apparatus and further embodiments of the further medical imaging apparatus 30 are conceivable, such as an X-ray apparatus, etc.

The magnetic resonance apparatus 20 has a scanner unit embodied as a magnetic unit 21. The magnetic unit 21 comprises a main magnet 22, a gradient coil unit 23, and a radio-frequency antenna unit 24. The main magnet 22 of the magnetic unit 21 is embodied to generate a strong and, in particular, constant main magnetic field 25. The main magnet 22 can be embodied, for example, as a superconducting main magnet 22 or also as a permanent magnet. The gradient coil unit 23 of the magnetic unit 21 is embodied to generate magnetic field gradients which are used for spatial encoding during imaging. The gradient coil unit 23 is controlled via a gradient control unit 26 of the magnetic resonance apparatus 20. The radio-frequency antenna unit 24 of the magnetic unit 21 is embodied to excite a polarization, which sets-in in the main magnetic field 25 generated by the main magnet 22. The radio-frequency antenna unit 24 is controlled by a radio-frequency antenna control unit 27 of the magnetic resonance apparatus 20, and irradiates radio-frequency magnetic resonance sequences into a patient-receiving region 11.

For controlling the main magnet 22, the gradient control unit 26 and for controlling the radio-frequency antenna control unit 27, the magnetic resonance apparatus 20 has a magnetic resonance control unit 28. The magnetic resonance control unit 28 centrally controls the magnetic resonance apparatus 20, such as carrying out a predetermined imaging gradient echo sequence. In addition, the magnetic resonance control unit 28 comprises an evaluation unit (not represented) for evaluating medical magnetic resonance image data.

The represented magnetic resonance apparatus 20 can of course comprise further components in which magnetic resonance apparatuses 20 customarily have. A general mode of operation of a magnetic resonance apparatus 20 is known to a person skilled in the art, moreover, so a detailed description of the general is omitted.

The second medical imaging apparatus, described herein by way of example and not limitation as a PET apparatus, comprises a scanner unit 31 with a plurality of Positron Emission Tomography detector modules 32 (PET detector modules 32), which are arranged into a ring shape and surround the patient-receiving region 11 in the circumferential direction. The PET detector modules 32 each have a plurality of Positron Emission Tomography detector elements (PET detector elements; not represented) which are arranged into a PET detector array which comprises a scintillation detector array with scintillation crystals, for example LSO crystals. Furthermore, the PET detector modules each comprise a photodiode array, for example Avalanche photodiode array or APD photodiode array, which are arranged inside the PET detector modules 32 downstream of the scintillation detector array.

Photon pairs, which result from the annihilation of a positron with an electron, are captured by means of the PET detector modules 32. Trajectories of the two photons enclose an angle of 180°. In addition, the two photons each have an energy of 511 keV. The positron is emitted here by a radiopharmaceutical, with the radiopharmaceutical being administered to the patient via an injection. The PET photons developed during the annihilation can be attenuated on passing through material in the beam path, with the attenuation probability depending on the path length through the material and the corresponding attenuation coefficient of the material.

In addition, the PET detector modules 32 each have an electronic detector device (not represented) which comprises an electric amplifier circuit and further electronic components (not represented).

For controlling the electronic detector device and the PET detector modules 32, the PET apparatus has a PET control unit 33. The PET control unit 33 centrally controls the PET apparatus. In addition, the PET control unit 33 comprises an evaluation unit for evaluating captured PET data.

The represented PET apparatus can of course comprise further components which PET apparatuses customarily have. A general mode of operation of a PET apparatus is known to a person skilled in the art, moreover, so a detailed description of the general components is omitted.

In the present exemplary embodiment, the two medical imaging apparatuses 30 are embodied in such a way that the PET scanner unit 31 of the second medical imaging apparatus 30 is integrated in the scanner unit, e.g. the magnetic unit 21, of the magnetic resonance apparatus 20. There is thus a single device with a single patient-receiving region 11 available for an examination of the patient, e.g. of the region of the patient to be examined.

The combined imaging apparatus 10 includes the patient-receiving region 11 for recording the patient for a combined MR-PET examination (in this example) on the patient. The patient-receiving region 11 in the present exemplary embodiment is cylindrical and cylindrically surrounded in a circumferential direction by the magnetic unit 21 and the scanner unit 31 of the PET apparatus. However, different embodiments of the patient-receiving region 11 are also conceivable. The combined imaging apparatus 10 has an enclosure 19 cylindrically surrounding the patient-receiving region 11.

For a positioning of the patient, e.g. a region of the patient to be examined, inside the patient-receiving region 11, the combined imaging apparatus 10 includes a patient-supporting apparatus 13. The patient-supporting apparatus 13 has a base unit 14 and a patient table 15, which can move in respect of the base unit 14. The patient table 15 is embodied to move inside the patient-receiving region 11 for positioning the patient, in particular the region of the patient to be examined. In particular, the patient table 15 is mounted so it can move in the direction of a longitudinal extension 16 of the patient-receiving region 11 and/or in the z-direction.

The combined imaging apparatus 10, e.g. the MR-PET apparatus, also has a central computing unit 17 which coordinates, for example, the capture and/or evaluation of magnetic resonance signals and PET signals. The central computing unit 17 can be a central system control unit.

Furthermore, the combined imaging apparatus 10, e.g. the MR-PET apparatus, comprises a user interface 18 that is connected to the central computing unit 17. Items of control information, such as image data, can be displayed for a medical operator on an output unit (not represented), for example on at least one monitor, of the user interface 18. Furthermore, the user interface 18 has an input unit (not represented) by means of which the medical operator can input items of information and/or parameters during a measuring process.

For a PET examination, it is customary to provide an attenuation correction map to take into account a loss of energy of the photons when capturing PET events, e.g. the photons. Exact capture of a position of the patient table 15 is necessary for this, with the patient table entering into the determination of an attenuation correction and/or the attenuation correction map. The combined imaging apparatus 10 has for this purpose a position-determining unit 40 (also referred to herein as position-determining system, which may include one or more sensors such as a capturing unit as described herein) which is embodied to determine a position of the patient table 15 in at least one direction. In the present exemplary embodiment, the position-determining unit 40 is embodied to determine the position of the patient table in three directions, e.g. in the x-direction, in the y-direction, and in the z-direction, which may be orthogonally oriented to one another. In an alternative embodiment, the position-determining unit 40 may be embodied to determine only the position of the patient table 15 in a single direction, e.g. in the z-direction, or also in two directions, e.g. in the z-direction and one further direction.

For determining the position of the patient table 15, the position-determining unit 40 has at least one capturing unit 41 (e.g. one or more sensors as discussed herein). In the present exemplary embodiment, the position-determining unit 40 has a single capturing unit 41 (also referred to herein as one or more sensors) (FIG. 1). In an alternative embodiment, the position-determining unit 40 may comprise two or three capturing units 41. The capturing unit 41 may be arranged on the enclosure 19 surrounding the patient-receiving region 11. The capturing unit 41 may be embodied to capture position information of the patient table 15 in at least one direction. In the present exemplary embodiment, the position information of the patient table 15 is arranged on a lower side 42 of the patient table 15. For this, the position-determining unit 40 has a position information element 43 that is arranged on the lower side 42 of the patient table 15.

The capturing unit 41 has a capture range 44 that is directed at the lower side 42 of the patient table 15. In the present exemplary embodiment, the capturing unit 41 has a camera (not represented). Alternatively or additionally, the capturing unit 41 can have further units, which appear expedient to a person skilled in the art, for capturing the position information and/or for capturing position data of the patient table 15.

For an arrangement of the capturing unit 41 inside the patient-receiving region 11, the position-determining unit 40 has a shielding unit 45 (also referred to herein as a shielding system). The shielding unit 45 comprises a shielding housing 46 with an optically transparent viewing window 47. The shielding housing 46 shields the capturing unit 41 with respect to radio-frequency radiation. The optically transparent viewing window 47 is arranged on a side of the shielding housing 46 that faces the patient-receiving region 11 and/or the patient table 15. In an embodiment, the optically transparent viewing window 47 also has properties which are shielding with respect to radio-frequency radiation. The optically transparent viewing window 47 may advantageously comprise a shatter-proof glass. For the shielding property, the optically transparent viewing window 47, e.g. the glass, may have an electrically conductive and transparent coating. For example, a coating of this kind of the glass can comprise an ITO material. Alternatively or additionally, it is also conceivable that the optically transparent viewing window 47 comprises a coating, for example a thin conductive layer made of silver.

In addition, the optically transparent viewing window 47 may be arranged inside the patient-receiving region 11 inclined in respect to a horizontal plane. For such an arrangement of the optically transparent viewing window 47, the shielding housing 46 may also be arranged and/or embodied in such a way that a side of the shielding housing 46 comprising the optically transparent viewing window 47 is also arranged inclined with respect to the horizontal plane. In an embodiment, the optically transparent viewing window 47 is arranged inside the patient-receiving region 11 inclined by any suitable angle such as, for instance, at least 10°, at least 15°, at least 18°, at least 20°, at least 22°, etc., with respect of the horizontal plane.

The shielding unit 45 may also have a protective cover 48 that is embodied to protect the optically transparent viewing window 47, and the capturing unit 41 may be arranged inside the shielding housing 46. The protective cover 48 is arranged on the shielding housing 46 so it can move. The protective cover 48 is arranged on the shielding housing 46 so it can move between two end positions. In the first end position, the protective cover 48 is situated above the optically transparent viewing window 47, so the optically transparent viewing window 47 is covered by the protective cover 48. In an embodiment, the optically transparent viewing window 47 is completely covered by the protective cover 48 in the first end position. In the second end position, in the direction of movement 49 of the protective cover 48, the protective cover 48 is situated next to the optically transparent viewing window 47, so the optically transparent viewing window 47 is uncovered and the capture range 44 of the capturing unit 41 arranged inside the shielding housing 46 is completely free of the protective cover 48.

The protective cover 48 is embodied to be compatible with magnetic resonance, for example the protective cover 48 comprises a magnetic resonance-compatible polymer material. In addition, the protective cover has a cleaning lip 50. The cleaning lip 50 is arranged on an edge 51 of the protective cover 48 that faces the optically transparent viewing window 47, with the edge 51 being transversely oriented to the direction of movement 49 of the protective cover 48.

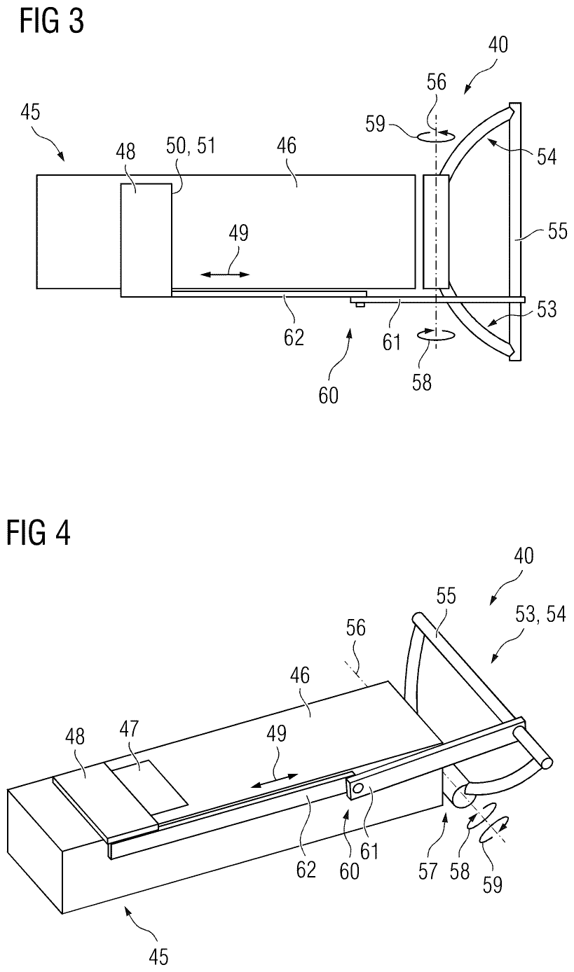

For a movement of the protective cover 48, the shielding unit 45 has a magnetic resonance-compatible actuator unit 53 (also referred to herein simply as a magnetic resonance-compatible actuator; see FIG. 2 to 4). The magnetic resonance-compatible actuator unit 53 is embodied to generate a drive moment for the movement of the protective cover 48 on the shielding housing 46. The magnetic resonance-compatible actuator unit 53 has a magnetic resonance-compatible stepper motor 54 with an actuator coil 55.

The actuator coil 55 comprises a plurality of windings made of copper wire, which may be arranged embedded in a housing made of plastics material. The actuator coil 55 has the form of a circle segment. A circle with a radius may be assigned to the circle segment of the actuator coil 55, with the radius of the circle of the circle segment being smaller than a radius of a circular cross-sectional area of the patient-receiving region 11. The actuator coil 55 may be e.g. shaped in such a way and/or have a size that with a movement, e.g. a rotation, of the actuator coil 55 about an axis of rotation 56, the actuator coil 55 does not touch and/or have any contact with the enclosure 19 surrounding the patient-receiving region 11. In an embodiment, the value of the radius of the circle of the circle segment may comprise any suitable proportion of the value of the radius of the patient-receiving region 11, such as for instance at most 50%, between 25% and 50%, etc., of the value of the radius of the patient-receiving region 11. The circle segment of the actuator coil 55 may e.g. comprise a central angle of at most, 90°.

The magnetic resonance-compatible actuator unit 55 also has an axis of rotation 56 about which the actuator coil 55 is rotatably arranged. To generate the drive moment, the actuator coil 55 executes a rotational movement about the axis of rotation 56. The axis of rotation 56 is oriented perpendicular to a direction of movement 49 of the protective cover 48. In addition, the axis of rotation 56 is oriented parallel to an x-axis of the magnetic resonance apparatus 20. The axis of rotation 56 is arranged on the shielding housing 46. The axis of rotation 56 is arranged at an edge region 57 of the shielding housing 46, with the edge region 57 being arranged on the shielding housing 46 at an edge of the shielding housing 46 which faces the enclosure 19 surrounding the patient-receiving region 11.

The magnetic resonance-compatible stepper motor 54 is embodied as an electric motor, with a stator of the magnetic resonance-compatible stepper motor comprising the main magnetic field 25 of the magnetic resonance apparatus 20. By applying an electric voltage to the actuator coil 55, a movement, e.g. a rotational movement, of the actuator coil 55 about the axis of rotation 56 is induced in a first direction of rotation 58 owing to a Lorentz force acting on the actuator coil 55. A change in the current direction of an electric current flowing through the actuator coil 55 can induce a rotational movement of the actuator coil 55 about the axis of rotation 56 in a second direction of rotation 59. The second direction of rotation 59 of the actuator coil 55 is oriented counter to the first direction of rotation 58 of the actuator coil 55. For setting a current direction in the actuator coil 55, the magnetic resonance-compatible actuator unit 53 has an H-bridge circuit (not represented).

The magnetic resonance-compatible actuator unit 53 also has a force transfer unit 60 which transfers a movement generated by the magnetic resonance-compatible actuator unit 53 to the protective cover 48 for a movement of the protective cover 48 on the shielding housing 46. In the present exemplary embodiment, the force transfer unit 60 has two force transfer elements 61, 62, with each of the two force transfer elements 61, 62 being embodied as a joint rod in the present exemplary embodiment. In alternative embodiments, the individual force transfer elements 61, 62 may differ from a joint rod. The two joint rods are arranged at the side of the shielding housing 46. A first joint rod is connected to a region of the actuator coil 55 by means of a first joint of the force transfer unit 60, with the region of the actuator coil 55 being arranged on the actuator coil 55 opposite the axis of rotation 56. The first joint rod is connected to the second joint rod by means of a second joint of the force transfer unit 60. The second joint rod may be permanently connected to the protective cover 48. The second joint rod may be oriented parallel to the direction of movement 49 of the protective cover 48.

The position-determining unit 40 also has a control unit 63, which may also be referred to herein as a controller, a control system, or control circuitry. The control unit 63 may be arranged outside of the patient-receiving region 11 and is shown in FIG. 1. Data may be e.g. transferred between the capturing unit 41 and the control unit 63 and, therewith, data may be transferred between a region inside the patient-receiving region 11 and a region outside of the patient-receiving region 11 via cables. The cables may comprise, for example, fiber optic cables and/or further cables which appear expedient to a person skilled in the art.

The control unit 63 is embodied to control a movement of the protective cover 48 on the shielding housing 46. The control unit 63 actuates the H-bridge circuit of the magnetic resonance-compatible actuator unit 53. In an embodiment, the control unit 63 coordinates a position of the protective cover 48 with capture of items of position information and/or of position data of the patient table 15 by means of the capturing unit 41. The control unit may include appropriate software and/or computer programs for this purpose.

Furthermore, the control unit 63 may be embodied to evaluate the position data and/or items of position information of the patient table 15 captured by the capturing unit 41. For this, the control unit 63 may e.g. include an evaluation unit (not represented, but alternatively referred to herein as evaluation circuitry). The evaluation unit also has an evaluation algorithm which can detect and/or determine a state (e.g. dirtying) of the optically transparent viewing window 47 on the basis of the captured position data and/or the captured items of position information of the patient table 15. For example, ideal position data with an ideally clean optically transparent viewing window 47 may be stored in the control unit 63, e.g. in the evaluation unit, and the evaluation unit may then establish and determine a degree of dirtying of the optically transparent viewing window 47 by way of a comparison of position data currently being captured with the ideal position data.

Furthermore, when dirtying of the optically transparent viewing window 47 is detected (e.g. the comparison identifies a dirtying in excess of a predefined threshold), the control unit 63 is embodied to initiate a cleaning process by way of a movement of the protective cover 48. In this embodiment, the magnetic resonance-compatible actuator unit 53 is actuated by the control unit 63 via the H-bridge circuit in such a way that the protective cover 48 (with the cleaning lip 50) carries out at least one forwards movement and one backwards movement, e.g. a plurality of forward movements and a plurality of backward movements, in the direction of movement 49 of the protective cover 48 on the optically transparent viewing window 47. The control unit 63 may carry out such a cleaning processes automatically after detection of such a dirtying of the optically transparent viewing window 47, with a trigger event for the cleaning process comprising detection of the dirtying of the optically transparent viewing window 47. Alternatively, the control unit 63 may be embodied to start the cleaning process when requested by the user, e.g. by the medical operator.

Alternatively or additionally, the control unit 63 may be embodied to generate user information with respect to the dirtying and to provide it for output to a user, e.g. to the medical operator via the user interface 18, when the control unit 63 detects dirtying of the optically transparent viewing window 47. The user information may comprise for instance information with regard to the dirtying of the optically transparent viewing window 47. In addition, the user information may comprise information about a current cleaning process automatically being carried out by the control unit 63. In addition, the user information may comprise cleaning information that offers the user, e.g. the medical operator, a proposal for a cleaning process for cleaning the optically transparent viewing window 47. The proposal for a cleaning process can comprise automatic cleaning of the optically transparent viewing window 47 controlled by the control unit 63. In addition, the proposal for a cleaning process can also comprise manual cleaning by the user, e.g. the medical operator.

Again, the user information may be output to the medical operator via the user interface 18, e.g. an output unit of the user interface 18. Selection and/or confirmation of a proposal for a cleaning process of the optically transparent viewing window 47 by the user (e.g. the medical operator) may also take place via the user interface 18, e.g. via an input unit of the user interface 18.

Although the disclosure has been illustrated and described in detail by the preferred exemplary embodiment, it is not limited by the disclosed examples and a person skilled in the art can derive other variation herefrom without departing from the scope of the disclosure.

Independent of the grammatical term usage, individuals with male, female or other gender identities are included within the term.

Additionally, the various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently or configured to execute instructions or computer programs that are stored on a suitable computer-readable medium. Regardless of the particular implementation, such units, etc., as applicable and relevant, may alternatively be referred to herein as “circuitry,” “controllers,” “processors,” or “processing circuitry,” or alternatively as noted herein.

What is claimed is:

1. A combined imaging apparatus, comprising:
a first medical imaging apparatus comprising a magnetic resonance apparatus;
a second medical imaging apparatus;
a patient-receiving region;
a patient-supporting apparatus including a patient table configured to be moved in at least one direction; and
a position-determining system including one or more sensors,
wherein the position-determining system is configured to determine a position of the patient table in the at least one direction, and
wherein the position-determining system comprises a shielding system including a shielding housing having an optically transparent viewing window and a protective cover for the optically transparent viewing window.

2. The combined imaging apparatus as claimed in claim 1, wherein the protective cover is arranged to move on the shielding housing.

3. The combined imaging apparatus as claimed in claim 2, wherein:
the protective cover is configured to move between a first end position and a second end position,
in the first end position, the protective cover covers the optically transparent viewing window, and
in the second end position, the optically transparent viewing window is not covered by the protective cover.

4. The combined imaging apparatus as claimed in claim 1, wherein the protective cover comprises a cleaning lip arranged on an edge of the protective cover that faces the optically transparent viewing window.

5. The combined imaging apparatus as claimed in claim 1, wherein the shielding system comprises a magnetic resonance-compatible actuator configured to generate a drive moment to move the protective cover on the shielding housing.

6. The combined imaging apparatus as claimed in claim 5, wherein the magnetic resonance-compatible actuator comprises a magnetic resonance-compatible stepper motor having an actuator coil configured to move, within a magnetic field of the magnetic resonance apparatus and in response to an electric current flowing through the actuator coil, the protective cover.

7. The combined imaging apparatus as claimed in claim 6, wherein the actuator coil has a form of a circle segment associated with a circle having a radius that is smaller than a radius of a circular cross-sectional area of the patient-receiving region.

8. The combined imaging apparatus as claimed in claim 6, wherein the actuator comprises an axis of rotation about which the actuator coil executes a rotational movement to generate the drive moment, and
wherein the axis of rotation is oriented parallel to an x-axis of the magnetic resonance apparatus and/or perpendicular to a direction of movement of the protective cover.

9. The combined imaging apparatus as claimed in claim 8, wherein the axis of rotation is arranged at an end region of the shielding housing, and
wherein the axis of rotation is arranged on the shielding housing at an edge region of the shielding housing that faces an enclosure surrounding the patient-receiving region.

10. The combined imaging apparatus as claimed in claim 1, wherein the position-determining system includes a controller configured to control a movement of the protective cover.

11. The combined imaging apparatus as claimed in claim 10, wherein the controller is configured to detect a state of the optically transparent viewing window based on captured position data and/or captured items of position information of the patient table.

12. The combined imaging apparatus as claimed in claim 11, wherein the controller is configured to initiate a cleaning process via a movement of the protective cover in response to the detected state of the optically transparent viewing window.

13. The combined imaging apparatus as claimed in claim 11, wherein the controller is configured to generate and output user information to a user interface in response to the detected state of the optically transparent viewing window.

14. The combined imaging apparatus as claimed in claim 1, wherein the optically transparent viewing window includes a glass cover with an electrically conductive coating.

15. The combined imaging apparatus as claimed in claim 1, wherein the optically transparent viewing window is inclined by at least 10° with respect to a horizontal plane of the patient-receiving region.
